(19) United States
(12) Patent Application Publication
(10) Pub. No.: US 2023/0200363 A1
(43) Pub. Date: Jun. 29, 2023

(54) TRANSGENIC FLUORESCENT ORNAMENTAL AMPHIBIANS

(71) Applicant: FireFrogs, Inc., Austin, TX (US)

(72) Inventor: Stephanie Testa, Austin, TX (US)

(73) Assignee: FireFrogs, Inc., Austin, TX (US)

(21) Appl. No.: 18/085,601

(22) Filed: Dec. 21, 2022

Related U.S. Application Data
(60) Provisional application No. 63/293,291, filed on Dec. 23, 2021.

(51) Int. Cl. A01K 67/027

(52) U.S. Cl. CPC A01K 67/0275; A01K 2227/50; A01K 2267/0393

(57) ABSTRACT

The present invention relates to the method and use of fluorescent proteins in making transgenic fluorescent ornamental amphibians. The fluorescent ornamental amphibians are used to establish a population of transgenic ornamental amphibians and to provide to the ornamental amphibian industry. Thus, new varieties of ornamental amphibians of different fluorescence colors from a novel source are developed.

### FIELD OF THE EMBODIMENTS

The field of the invention and its embodiments relate to transgenic gene constructs with gene promoters and heterologous genes for the generation of transgenic amphibians, and specifically, transgenic fluorescent ornamental amphibians.

### BACKGROUND OF THE EMBODIMENTS

Transgenic technology allows for the introduction of new and functional genetic material into the germ line. Specifically, transgenesis is a procedure that introduces an exogenous DNA, called a transgene, into the genome of a living organism. Alternatively, the transgene can be introduced into germ cells that are used for fecundation. The transgenic organism will exhibit a new property and will transmit it to its offspring.

As an example, mice have proven amenable to such genetic modification. (Gordon, et al., 1980) (Gordon, 1981). Subsequently, a variety of species, including fish and fruit flies, have been subject to transgenic manipulation. Germline gene transfer has increased the rate of progress in understanding mammalian development and has provided important insights into human diseases. (Palmiter & Brinster, 1986). (Lathe & Mullins, 1993). Ways to introduce a foreign gene into amphibians include, among others: microinjection (Du et al., 1992), electroporation (Powers et al., 1992), sperm-mediated gene transfer (Sin et al., 1993), gene bombardment or gene gun (Zelenin et al., 1991), liposome-mediated gene transfer (Szelei et al., 1994), and the direct injection of DNA into muscle tissue (Xu et al., 1999).

Green fluorescent protein (GFP) is a useful tool in the investigation of various cellular processes. Researchers have isolated the GFP gene from the jelly-fish Aqueous victoria. More recently, various other new fluorescent protein genes have been isolated from the Anthozoa class of coral reefs called DsRed, red fluorescent protein gene; ZsGreen, green fluorescent protein gene and ZsYellow, yellow fluorescent protein gene. (Matz, et al., 1999). The novel fluorescent proteins encoded by these genes share 26-30% identity with GFP. These bright fluorescent proteins each emit a distinct wavelength and are physico-chemically stable and versatile.

Fluorescent proteins have broad application in research and development. The red fluorescent protein, DsRed, has been used as a reporter in the transgenic studies involving various animal model systems, such as: filamentous fungi (Eckert et al., 2005), ascidian (Zeller et al., 2006), zebrafish (Zhu et al., 2005 and Zhu et al., 2004), Xenopus (Werdien et al., 2001), insect (Cho et al., 2006), and plants (Wenck et al., 2003), as well as a marker in imaging studies in stem cells (Tolar et al., 2005). The green fluorescent protein, ZsGreen, has been used as a transformation marker in insects (Sarkar et al., 2006), knock-in mouse model for the study of KIT expressing cells (Wouters et al., 2005), and as reporters for plant transformation (Wenck et al., 2003). Additionally, the yellow fluorescent protein, ZsYellow, has been used a reporter for plant transformation (Wenck et al., 2003) and for visualizing fungal pathogens. Despite the promising results of these transgenic experiments, methods and systems of using transgenic gene constructs with gene promoters and heterologous genes for the generation of transgenic fluorescent ornamental amphibians are needed.

Examples of related art include:

U.S. Pat. No. 10,798,923 B2 describes transgenic blue ornamental fish, as well as methods of making such fish by in vitro fertilization techniques.

U.S. Published Patent Application No. 2003/0221206 A1 relates to methods for producing transgenic animals. Specifically, the methods of this reference include production of a transgenic animal by transgenic intracytoplasmic sperm injection, retroviral gene transfer, intracytoplasmic nuclear injection, and pronuclear injection. In addition, this reference also relates to methods for using transgenic animals as models for human disease and diagnosis.

U.S. Published Patent Application No. 2010/0037331 A1 relates to the method and use of reef coral fluorescent proteins in making transgenic red, green, and yellow fluorescent zebrafish. Preferably, such fluorescent zebrafish are fertile and are used to establish a population of transgenic zebrafish.

WO03102176A1 relates to transgenic aquatic animals, particularly the clawed frog and the zebra fish and cells derived therefrom, characterized in comprising at least one expression cassette with a regulatory DNA sequence selected from the response elements to nuclear hormone receptors, particularly TRE, connected in a functional manner downstream of a DNA segment coding for a marker protein such as luciferase or GFP.

WO03022040A2 relates to methods for producing transgenic animals using retroviral constructs engineered to carry one or more transgenes of interest.

Some similar methods are known in the art. However, their means of operation are substantially different from the present disclosure, as the other inventions fail to solve all the problems taught by the present disclosure.

### SUMMARY OF THE EMBODIMENTS

The present invention and its embodiments relate to transgenic gene constructs with gene promoters and heterologous genes for the generation of transgenic amphibians, and specifically, transgenic fluorescent ornamental amphibians.

A first embodiment of the present invention describes a transgenic fluorescent ornamental amphibian comprising, in its genome, a transgene encoding a fluorescent protein. As described herein, a “transgene” is genetic material that has been transferred by genetic engineering techniques from one organism to another. In examples, the “transgene” may also contain synthetic DNA sequences, such as a codon-optimized sequence specific to amphibians or specific to the species of amphibian being modified. Further, the final product may include one or more fluorescent proteins, produced by either one or more rounds of modification.

In examples, the fluorescent proteins are ZsGreen1, ZsYellow1, DsRed2, GFP, eGFP, YFP, eYFP, BFP, eBFP, CFP, eCFP, FP, AmCyan1, DsRed-Express, AsRed2, HcRed1, mPlum, mCherry, tdTomato, mStrawberry, J-Red, DsRed-monomer, morange, mKO, MCitrine, Venus, Ypet, EYFP, Emerald, CyPet, mCFPm, Cerulean, or T-Sapphire, among others not explicitly listed herein.

The transgene is under the control of a ubiquitous promoter, such as: acidic ribosomal phosphoprotein (ARP), α-catenin, β-catenin, γ-catenin, or EF-1 α, among others not explicitly listed herein; and/or the transgene is under the control of a tissue specific promoter. In examples, the tissue specific promoter is a muscle-specific promoter. In specific examples, the muscle-specific promoter is β-actin, desmin, dystrophin, myosin heavy chain, myosin light chain, MyoD, myogenin, muscle creatine kinase, β-sarcoglycan, serum response factor, or α-tropomyosin, among others not explicitly listed herein. In other examples, the tissue specific promoter comprises a skin specific promoter, an eye specific promoter, or a bone specific promoter, among others not explicitly listed herein.

A species of the transgenic fluorescent ornamental amphibian is Hymenochirus, sp., Hymenochirus boettgeri, Hymenochirus boulengeri, Hymenochirus curtipes, Hymenochirus feae, Xenopus laevis, Trachycephalus Resinfictrix, Anaxyrus americanus, Ambystoma mexicanum, Lepidobatrachus laevis, Melanophryniscus stelzneri, Typhlonectes compressicauda, Xenopus, sp., Triturus cristatus, Bombina, sp., Cynops, sp., Salamandra, Cryptobranchidae, Hyla versicolor, Occidozyga lima, Cryptobranchus alleganiensis, Neurergus kaiseri, Siren intermedia, Necturus, sp., Ceratophryidae, sp., Lithobates grylio, Pyxicephalus, sp., Dendrobates, sp., Agalychnis callidryas, Typhlonectes natans, Ambystoma tigrinum, Xenopus tropicalis, or Lictoria caerulea, among others not explicitly listed herein.

A second embodiment of the present invention describes a method to produce a transgenic fluorescent ornamental amphibian. The method includes numerous process steps such as: obtaining or developing an initial cloning vector with a fluorescent gene, which is then used to assemble a final cloning vector with the complete transgene. The initial cloning vector comprises a gene encoding a fluorescent protein. The fluorescent gene is replicated in bacteria transformed with a cloning vector, such as pUC19, among others not explicitly listed herein.

The final cloning vector contains a complete transgene encoding a promotor that promotes transcription of the fluorescent gene in amphibians, one or more fluorescent genes that transcribe one or more fluorescent proteins, and one or more polyadenylation signals, wherefrom the transgene will be isolated and used to develop modified amphibians. The transgene is under the control of a ubiquitous promoter and/or a tissue specific promoter.

In examples, the ubiquitous promoter is acidic ribosomal phosphoprotein (ARP), α-catenin, β-catenin, 7-catenin, or EF-1 α, among others not explicitly listed herein. In examples, the tissue specific promoter is a muscle-specific promoter. In preferred examples, the muscle-specific promoter is β-actin, desmin, dystrophin, myosin heavy chain, myosin light chain, MyoD, myogenin, muscle creatine kinase, β-sarcoglycan, serum response factor, or α-tropomyosin, among others not explicitly listed herein.

In some examples, the complete cloning vector comprises one polyadenylation signal. In other examples, the complete cloning vector comprises at least two polyadenylation signals positioned in tandem. In other examples, at least one or two polyadenylation signals are viral polyadenylation signals. Further, in some preferred examples, at least one or two polyadenylation signals are SV40 polyadenylation sequences.

A third embodiment of the present invention describes a method of producing a population of transgenic fluorescent ornamental amphibians. One or more amphibians of the population of the transgenic fluorescent ornamental amphibians comprise and express one or more transgenes from the cloning vectors in its genome, which express one or more fluorescent proteins.

### BRIEF DESCRIPTION OF THE DRAWINGS

FIG. 1 depicts a schematic map of a cloning vector containing a transgenic construct, according to at least some embodiments disclosed herein.

FIG. 2 depicts a schematic diagram of a gene construct development and preparation process, according to at least some embodiments disclosed herein.

FIG. 3 depicts a schematic diagram of amphibian modification using a transgenesis procedure, according to at least some embodiments disclosed herein.

FIG. 4 depicts images of glow in pigmented as compared to albino amphibians, according to at least some embodiments disclosed herein.

FIG. 5 depicts a chart comparing traits between a model organism and another organism, Hymenochirus boettgeri, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Transgenic Constructs Plasmids are convenient and efficient cloning vectors for carrying out a variety of recombinant DNA procedures. Generating a typical transgenic construct involves assembling three basic DNA elements: (1) a promoter and/or enhancer that confers the desired spatial and temporal pattern of transgene expression; (2) the gene to be transcribed, which may or may not encode a protein; and (3) a transcription termination or polyadenylation signal sequence to stop transcription and enable 3′ end processing. An optional fourth DNA element is a genomic boundary element for reducing position effects. The present invention encompasses transgenic constructs to produce a transgenic amphibian, and more specifically, a transgenic ornamental amphibian.

The manner of introduction and the structure of a transgenic construct renders such a transgenic construct an exogenous construct. Although a transgenic construct can be made up of any assembly of nucleic acid sequences, for use in the disclosed invention, it is preferred that the transgenic construct combine regulatory elements operably linked to a sequence encoding one or more proteins. The methods and protocols for designing and making transgenic constructs are well known to those skilled in the art. (Sambrook et al., 2001) (Sambrook et al., 1989).

The process to develop a transgenic amphibian having a predictable pattern of transgenic expression begins with creating a genetic construct. As explained, the genetic construct typically includes: transcriptional regulators comprising a promoter, a gene and appropriate RNA-processing and/or translational enhancing motif. The gene promoter determines where, when, and under what conditions the gene is expressed. The gene contains protein coding portions that determine the protein to be synthesized and thus the biological function. The gene might also contain intron sequences which can affect mRNA processing or which might contain transcription regulatory elements. The RNA processing signals may include: one or more polyadenylation signals and one or more introns. Among the three portions, it is preferable to use a promoter that drives strong expression. The promoter may be a homologous promoter or it may be a heterologous promoter.

It should be appreciated that a promoter drives expression predominantly in a tissue if expression is at least 2-fold, preferably at least 5-fold higher in that tissue compared to a reference tissue. A promoter drives expression specifically in a tissue if the level of expression is at least 5-fold, preferably at least 10-fold higher, more preferably at least 50-fold higher in that tissue than in any other tissue. A ubiquitous promoter drives expression in most tissues, and preferably in all tissues.

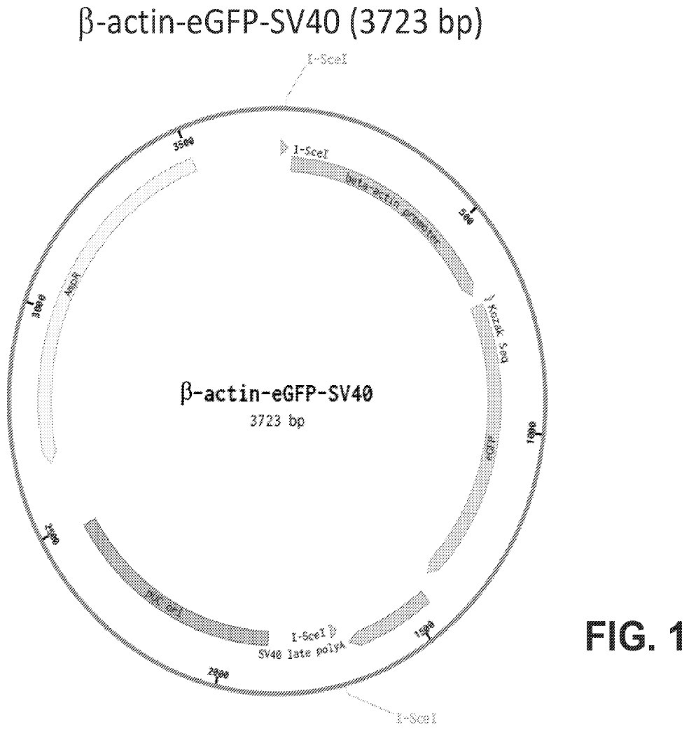

FIG. 1 depicts a schematic map of a transgenic construct of the present invention. More specifically, FIG. 1 depicts the schematic map of the transgenic construct β-actin-eGFP-SV40. Specifically, the transgenic construct includes a promoter (such as β-actin, a muscle-specific promotor), a fluorescent gene (such as enhanced green fluorescent protein or eGFP), and a terminator sequence (such as SV40-polyA), where the transgenic construct is flanked by I-SceI restriction enzyme sites. The I-SceI sites have been utilized in methods for generating transgenic Xenopus. (Ogino, et al., 2006). It should be appreciated that the promotor and the fluorescent gene sequences are interchangeable. It should also be appreciated that other restriction enzyme sites can be substituted in place of the I-SceI sites. Moreover, several different promoters and fluorescent genes can be used, which will be discussed further herein.

### Recombinant DNA Constructs

Recombinant DNA (rDNA) molecules are DNA molecules formed by methods of genetic recombination (such as molecular cloning) that bring together genetic material from multiple sources, creating sequences that would not otherwise be found in the genome. Recombinant DNA constructs that include one or more DNA sequences described herein and an additional DNA sequence are also included within the scope of this invention. The DNA sequences described as constructs or in vectors are “operably linked” with other DNA sequences. DNA regions are operably linked when they are functionally related to each other. Generally, operably linked means contiguous (or in close proximity to).

In some examples, the disclosed transgenic constructs preferably include other sequences that improve expression from, or stability of, the construct. In other examples described herein, the invention includes a polyadenylation signal in the construct that encodes a protein, which ensures that mRNA transcripts from the transgene will be efficiently translated as protein. The identification and use of polyadenylation signals in expression constructs is well established. In preferred examples, at least two polyadenylation signals may be used, and in some examples, these signals are two copies of SV40 polyadenylation sequence.

In certain examples, methods are provided that use multiple vectors to express at least one fluorescent protein in order to enhance expression. In some examples, the transgenic amphibian is created comprising, in its genome, one or more fluorescent transgenes under the control of ubiquitous and/or tissue specific promoters. As described herein, a ubiquitous promoter is strongly active in a wide range of cells, tissues, and cell cycles. Ubiquitous promoters are available as native or composite promoters. A minimal promoter or core promoter refers to the minimal sequence of a native promoter, which may be used to limit the total size of the transgenic construct. In other examples, a genetically engineered or synthetic promoter may be used, which would contain elements of promoters from more than one species.

A tissue-specific promoter is a promoter that has activity in only certain cell types. Use of a tissue-specific promoter in the expression cassette can restrict unwanted transgene expression as well as facilitate persistent transgene expression. In a preferred embodiment, the tissue specific promoter is muscle-specific. In some examples, the ubiquitous promoter and the muscle-specific promoter are selected from the model organism Xenopus and are presented in Table 1 below. However, it should be appreciated that other promoters may be used, which are not explicitly described herein. An extensive list of promoters with expression of interest can be found using NCBI protein database server (www.ncbi.nlm.nih.gov/sites/entrez?db=Protein).

TABLE 1
Promoters from Xenopus for transgene expression in amphibians

| Gene Promoter | Promoter Type |
|---|---|
| Acidic ribosomal | Ubiquitous |
| phosphoprotein (ARP) | |
| alpha-catenin | Ubiquitous |
| beta-catenin | Ubiquitous |
| gamma-catenin | Ubiquitous |
| EF-1 alpha | Ubiquitous |
| beta-actin | Muscle-specific |
| Desmin | Muscle-specific |
| Dystrophin | Muscle-specific |
| Myosin heavy chain | Muscle-specific |
| Myosin light chain | Muscle-specific |
| MyoD | Muscle-specific |
| Myogenin | Muscle-specific |
| Muscle creatine kinase | Muscle-specific |
| beta-sarcoglycan | Muscle-specific |
| Serum response factor | Muscle-specific |
| a-tropomyosin | Muscle-specific |

In some examples described herein, more than one construct containing different promoters can be injected into the unfertilized egg of the amphibian simultaneously. It is also a subject of this invention to disclose expression of one or more fluorescent protein gene specifically in chromatophores. Chromatophores are pigment-containing and light-reflecting cells found in animals. There are several types of chromatophores, such as: melanophores (black), xanthophores (yellow), erythrophores (red), cyanophores (blue), leucophores (white) and iridophores (reflective). Of those, only melanophores, called melanocytes, are found in higher vertebrates, such as mammals. Different species of amphibian contain all types of chromatophores.

These different cell types express specific genes characteristic only for them or specific for a subset of chromatophores. For example, tyrosinase-related protein 1 (tyrp1) is found only in melanophores and ednrb1 is found in malenocytes and iridophores. Promoters of these specific genes fused to fluorescent protein open reading frames (ORFs) can be used to visualize specific chromatophores. The specific genes can be divided into two groups: regulatory proteins and biosynthesis enzymes, involved in specific pigment synthesis (e.g., sepiapterin reductase, involved in yellow pigment synthesis in xanthophores). Expression of regulatory proteins usually is at a lower level than that of biosynthesis enzymes, and as such, use of promoters of biosynthesis enzymes are preferred.

Those having ordinary skill in the art understand that the presence of introns in primary transcripts can increase expression, possibly by causing the transcript to enter the processing and transport system for mRNA. It is preferred that the intron be homologous to the host species, and more preferably homologous to the expression sequences used (that is, that the intron be from the same gene that some or all of the expression sequences are from). (Palmiter et al., 1991) (Sippel et al., 1992) (Clark et al., 1993).

The heterologous fluorescent gene may be, for example, a gene encoding DsRed2, Orange2, AmCyan, ZsGreen1, and ZsYellow1. The heterologous fluorescent gene may also be any variation or mutation of these genes, encoding fluorescent proteins including: green fluorescent protein (GFP), enhanced green fluorescent protein (eGFP), yellow fluorescent protein (YFP), enhanced yellow fluorescent protein (eYFP), blue fluorescent protein (BFP), enhanced blue fluorescent protein (eBFP), cyan fluorescent protein (CFP) and enhanced cyan fluorescent protein (eCFP), any of the proteins available on https://www.fpbase.org/table/, any variation or mutation thereof, or any other fluorescence proteins.

### Substitutions, Additions and Deletions

In some examples described herein, the polypeptide may have additional individual amino acids or amino acid sequences inserted into the polypeptide in the middle, at the N-terminal, and/or at the C-terminal ends as long as the polypeptide possesses the desired physical and/or biological characteristics. Likewise, some of the amino acids or amino acid sequences may be deleted from the polypeptide as long as the polypeptide possesses the desired physical and/or biochemical characteristics. Amino acid substitutions may also be made in the sequences as long as the polypeptide possesses the desired physical and biochemical characteristics. DNA coding for these variants can be used to prepare gene constructs of the present invention.

A nucleic acid sequence “encodes” or “codes for” a polypeptide if it directs the expression of the polypeptide referred to. The nucleic acid can be DNA or RNA. Unless otherwise specified herein, a nucleic acid sequence that encodes a polypeptide includes the transcribed strand, the hnRNA, and the spliced RNA or the DNA representative thereof.

### Transgenic Amphibians

The disclosed constructs and methods can be used with any type of amphibian. It is preferred that the amphibian belong to a species and variety of commercial value, particularly one having commercial value within the ornamental amphibian industry. A list of ornamental amphibian species can be found in Table 2 below. However, it should be appreciated that other species of amphibians may be used, which are not explicitly listed or described herein.

TABLE 2
Ornamental Amphibian Species

| Scientific Name | Common Name |
|---|---|
| Hymenochirus, sp. | African Dwarf Frog (general name) |
| Hymenochirus boettgeri | Zaire/Congo African Dwarf Frog |
| Hymenochirus boulengeri | Eastern African Dwarf Frog |
| Hymenochirus curtipes | Western African Dwarf Frog |
| Hymenochirus feae | Gaboon African Dwarf Frog |
| Xenopus laevis | African Clawed Frog |
| Trachycephalus Resinfictrix | Amazon Milk Frog |
| Anaxyrus americanus | American Toad |
| Ambystoma mexicanum | Axolotl (salamander) |
| Lepidobatrachus laevis | Budgett's Frog |
| Melanophryniscus stelzneri | Bumble Bee Toad |
| Typhlonectes compressicauda | Cayenne Caecilian |
| Xenopus, sp. | Clawed Frog |
| Triturus cristatus | Crested Newt |
| Bombina, sp. | Fire-Bellied Toad |
| Cynops, sp. | Fire-Bellied Newt |
| Salamandra | Fire Salamander |
| Cryptobranchidae | Giant Salamander |
| Hyla versicolor | Gray Tree Frog |
| Occidozxga lima | Green Puddle Frog |
| Cryptobranchus alleganiensis | Hellbender (salamander) |
| Neurergus kaiseri | Kaiser's Mountain Newt |
| Siren intermedia | Lesser Siren (salamander) |
| Necturus, sp. | Mudpuppy |
| Ceratophryidae, sp. | Pacman Frog |
| Lithobates grylio | Pig Frog |
| Pyxicephalus, sp. | Pixie Frog (aka African Bull Frog) |
| Dendrobates, sp. | Poison Dart Frog |
| Agalychnis callidryas | Red-Eyed Tree Frog |
| Typhlonectes natans | Rubber Eel (caecilian) |
| Ambystoma tigrinum | Tiger Salamander |
| Xenopus tropicalis | Western Clawed Frog |
| Lictoria caerulea | Whites Tree Frog |

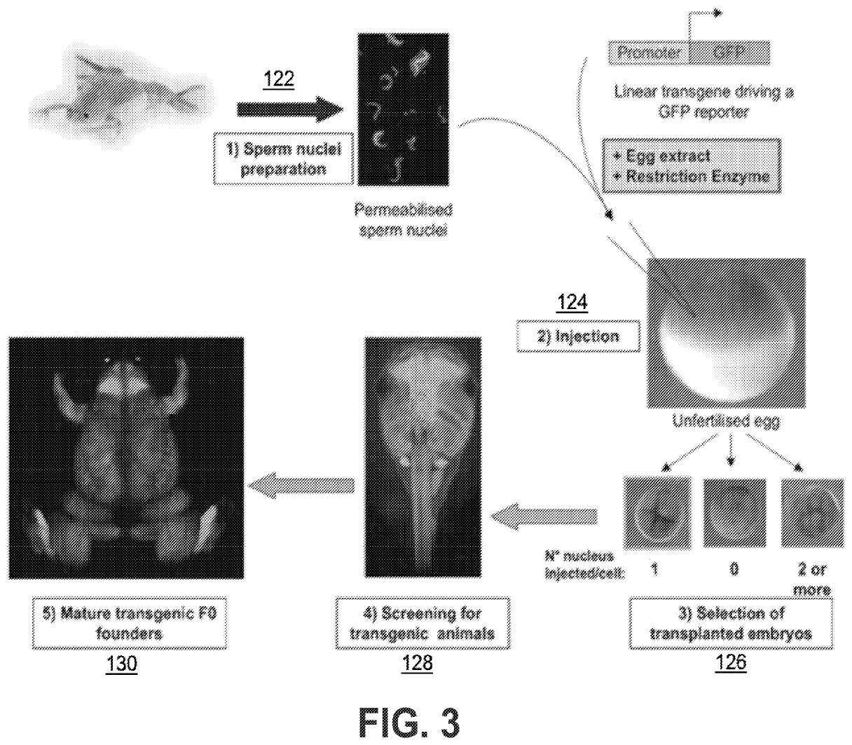

The disclosed transgenic amphibians are produced by introducing a transgenic construct into the genomes of cells of an amphibian, preferably embryonic cells, and most preferably in a single cell embryo or starting with a single cell oocyte. In some examples, microinjection may be done according to FIG. 3, where permeabilized sperm nuclei are microinjected into harvested oocytes with the gene construct in tandem, or harvested oocytes may be fertilized using sperm obtained from dissected testis immediately before microinjection of the gene construct.

Where the transgenic construct is introduced into embryonic cells, the transgenic amphibian is obtained by allowing the embryonic cell or cells to develop into an amphibian. The disclosed transgenic constructs can be introduced into embryonic amphibian cells using any suitable technique. Many techniques for such introduction of exogenous genetic material have been demonstrated in amphibians and other animals, which include: microinjection (Culp et al., 1991), electroporation (Inoue et al., 1990), particle gun bombardment (Zelenin et al., 1991), and the use of liposomes (Szelei et al., 1994). The preferred method for introduction of transgenic constructs into amphibian embryonic cells is by microinjection.

Embryos or embryonic cells can generally be obtained by collecting eggs as soon as possible after they are laid by methods that are well known to those of ordinary skill in the art. Depending on the amphibian, it is generally preferred that the eggs be fertilized prior to or at the time of collection. A fertilized egg cell prior to the first cell division is considered a one cell embryo, and the fertilized egg cell is thus considered an embryonic cell. However, in other examples, frozen amphibian sperm may be used to fertilize eggs. (Walker and Streisinger, 1983). Recently obtained fresh amphibian sperm may also be used to fertilize eggs (oocytes) prior to microinjection or in tandem with the microinjection.

The transgene may randomly integrate into the genome of the embryo in one or more copies (concatemers). It should be appreciated that a concatemer is a long continuous DNA molecule that contains multiple copies of the same DNA sequence linked in series. These polymeric molecules are usually copies of an entire genome linked end to end and separated by cos sites. After introduction of the transgenic construct, the embryo is allowed to develop into an amphibian. The amphibians that are injected as embryos are screened for the presence of the transgene, and the successfully modified amphibians are allowed to interbreed and the offspring are further screened for the presence of the transgene.

Amphibians harboring the transgene may be identified by any suitable means. In the preferred case, one or more of the transgenic constructs will have integrated into the cellular genome, which can be probed for the presence of construct sequences. To identify transgenic amphibians actually expressing the transgene, the presence of an expression product can be assayed. Several techniques are known to those having ordinary skill in the art for such identification. Probing of potential or actual transgenic amphibians for nucleic acid sequences present in or characteristic of a transgenic construct can be accomplished by Southern or northern blotting, polymerase chain reaction (PCR) or other sequence-specific nucleic acid amplification techniques known to those having ordinary skill in the art.

The simplest way to confirm the presence of a fluorescent protein expressing the transgene in a given amphibian is by visual inspection, as the amphibian in question would be brightly colored and immediately distinguishable from non-transgenic amphibians. One can observe all transgenic fluorescent amphibians from a particular population that exhibit strong visible fluorescence under the various lighting conditions and select the amphibian that exhibits the highest level of visible fluorescence of the fluorescent protein. The selected amphibian with the strong visible fluorescence may then be monitored and selected continuously to ensure stability of expression and maintenance of the strong visible fluorescence trait. Thus, a new line of amphibians exhibiting strong visible fluorescence may be created for further breeding.

The invention further includes progeny of the transgenic amphibians containing a genomically integrated transgenic construct, as well as transgenic amphibians derived from a transgenic egg, sperm cell, embryo, or other cell containing a genomically integrated transgenic construct. As described herein, “progeny” can result from breeding two transgenic amphibians of the present invention or can result from breeding a first transgenic amphibian of the present invention with a second non-transgenic amphibian (e.g., a wild-type amphibian, a specialized strain of amphibian, or a mutant amphibian). The hybrid progeny of these matings have the benefits of the transgene for fluorescence combined with the benefits derived from the other lineages.

### Xenopus and Hymenochirus

The African clawed frog, or Xenopus, is a species of African aquatic frog of the family Pipidae. The African clawed frog's name is derived from the three short claws on each hind foot, which it uses to tear apart its food. Transgenesis techniques have been used for Xenopus tropicalis, a model organism for developmental biology, based on a method described first for Medaka (or Oryzias latipes), which is a small, egg-laying freshwater teleost that is widely used as a laboratory animal. (Ogino, et al., 2006). This transgenic procedure includes co-injection of meganuclease I-SceI and a transgene construct flanked by two I-SceI sites into fertilized eggs. Researchers found that approximately 30% of injected embryos expressed transgenes in a promoter-dependent manner and about one-third of such embryos showed incorporation of the transgene at the one-cell stage and the remainder were “half-transgenics,” suggesting incorporation at the two-cell stage. (Ogino, et al., 2006). Transgenes from both classes of embryos have been shown to be transmitted and expressed in offspring. Researchers have further used this procedure in Xenopus laevis. (Ogino, et al., 2006).

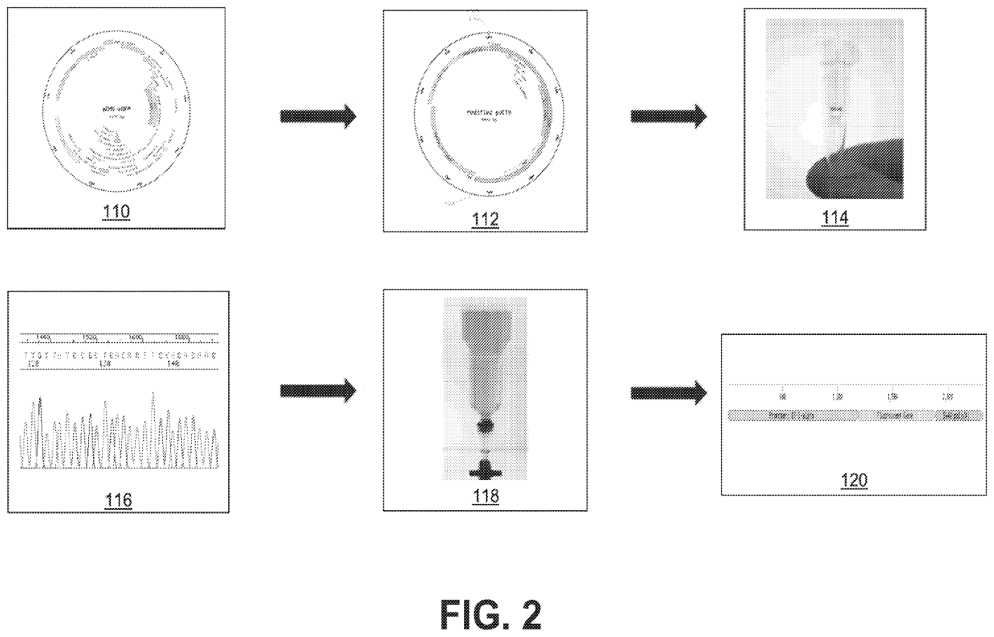

FIG. 2 depicts a schematic diagram of a gene construct development and preparation process, according to at least some embodiments disclosed herein. As shown in FIG. 2, the gene construct development and preparation process includes numerous steps, such as a first process step 110 that involves acquisition, isolation, and/or synthetic development of the fluorescent gene of interest, such as eGFP. The example of a possible starter plasmid can be found at https://www.addgene.org/11153/.

The second process step 112 includes synthesizing and optimizing the gene construct, where components of such construct may include: the promotor, the fluorescent gene, the SV40 terminator, and restriction enzyme sites, as described herein. This includes inserting the gene construct into a plasmid, such as the pUC19 plasmid. The pUC19 plasmid is a small, high-copy number E. coli plasmid cloning vector. The molecule is a small double-stranded circle, 2686 base pairs in length. pUC19 encodes the N-terminal fragment of b-galactosidase (lacZa), which allows for blue/white colony screening (e.g., α-complementation), ampicillin resistance (bla), as well as a pUC origin of replication.

A third process step 114 follows the second process step 112 and includes transforming E. coli and preparing the modified pUC19 plasmids. A fourth process step 116 follows the third process step 114 and includes validating the modified plasmids with DNA sequencing. It should be appreciated that this validation may occur by any method known to those having ordinary skill in the art.

A fifth process step 118 follows the fourth process step 116 and includes culturing and preparing the modified plasmids for scale-up. A sixth process step 120 follows the fifth process step 118 and includes lineralizing the DNA and preparing for microinjections. Though the SV40 terminator and pUC19 plasmid are described in FIG. 1 and FIG. 2, other terminators and plasmids known to those having ordinary skill in the art may be used herein.

FIG. 3 depicts a schematic diagram of amphibian modification using a transgenesis procedure, according to at least some embodiments disclosed herein. More specifically, FIG. 3 depicts the Restriction Enzyme Mediated Integration (REMI) transgenesis procedure for Xenopus taken from A. Chesneau et al., 2008, which has been incorporated by reference in its entirety. The REMI method of FIG. 3 includes numerous process steps, such as: a first process step 122, a second process step 124, a third process step 126, a fourth process step 128, and a fifth process step 130.

The first process step 122 of FIG. 3 includes isolating sperm nuclei, as described by Murray, 1991, with the modifications by Kroll and Amaya, 1996. The quality and concentration of the sperm solution are determined by Hoechst staining in a haemocytometer. The cells are then incubated with the linear transgene along with egg extract and restriction enzyme. Methods that aimed at improving the REMI method have discarded the use of egg extract and restriction enzyme, which made the method very similar to an ICSI protocol (Sparrow et al., 2000). The mixture is then injected into unfertilized eggs (e.g., the second process step 124) and the transplanted embryos are selected at the four-cell stage (e.g., the third process step 126).

All eggs are activated by the injection, but only the embryos that cleaved normally are isolated for further analyses. As the nuclei are injected at a constant flow rate, at best a third of the eggs are expected to receive a single nucleus. As development proceeds, embryos are scored for the expression of the transgene (e.g., the fourth process step 128) and placed into an husbandry facility to obtain mature FO founder animals to derive transgenic lines (e.g., the fifth process step 130). It should be appreciated that as described herein, the term “founder” refers to an individual with a genetic trait of interest that will be used to derive lines. The FOs are animals produced during the transgenesis procedure, for which the primary genetic trait of interest is the transgene.

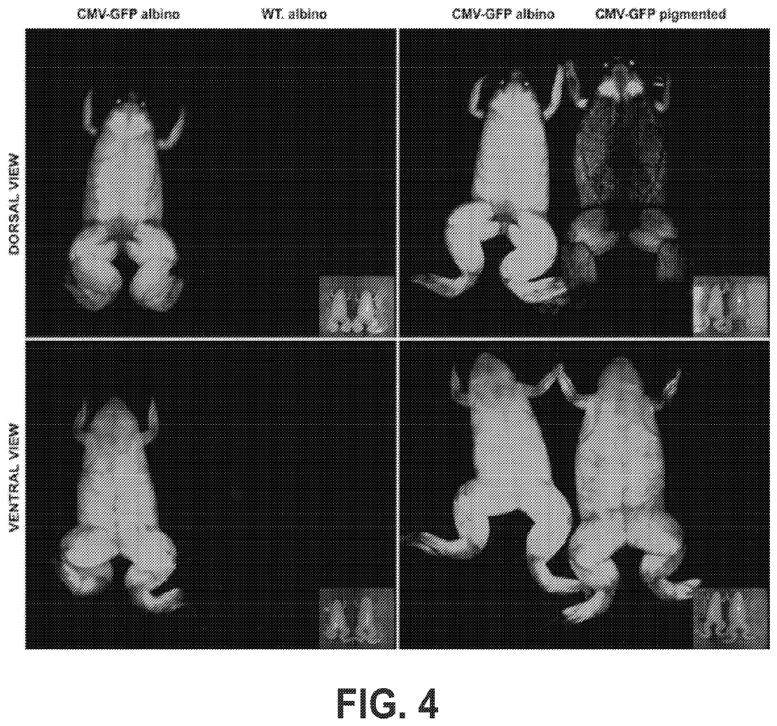

FIG. 4 depicts images of glow in pigmented as compared to albino amphibians taken from A. Chesneau et al., 2008, according to at least some embodiments disclosed herein. More specifically, GFP fluorescence was analyzed in an adult albino frog transgenic for a CMV (cytomegalovirus)-GFP reporter transgene that drives GFP expression ubiquitously. In the left-hand panels of FIG. 4, the transgenic albino frog is shown next to a wild-type albino animal, and in the right-hand panels next to a pigmented frog transgenic for the same construct. The pigments obscure the analyses of GFP expression on the dorsal side of the pigmented animal (e.g., the upper panels of FIG. 4). However, when observed on the ventral side, both the albino and pigmented animal express GFP to a similar extent (e.g., the lower panels of FIG. 4). Bright-field images are provided as insets in the main pictures.

In some examples, genetic modification may be used to create albino and leucistic amphibians, and populations therefrom, prior to the additional genetic modification steps used to develop fluorescence amphibians as described herein. This would occur in the case of white/golden variants that do not exist in the wild for some species of amphibians that one may be interested in modifying for fluorescence. For example, the existence of true albino Xenopus is well-known; however, true albino Hymenochirus have not been found in the wild and may not exist. The white/gold variants of Hymenochirus found on the market appear to be leucistic. While the modification of leucistic Hymenochirus may achieve a high enough degree of fluorescence for ornamental purposes, to develop a true albino fluorescent Hymenochirus, one would require additional modifications. For example, a first round of modification would involve the knocking out (disrupting the natural sequence) of a pigment gene or genes (for example, the TYR gene, which encodes for tyrosinase needed for the production of melanin), selection for the albino trait through visualization, and then a second round of modification of the albino progeny to incorporate the transgene containing a fluorescent gene or genes as described herein.

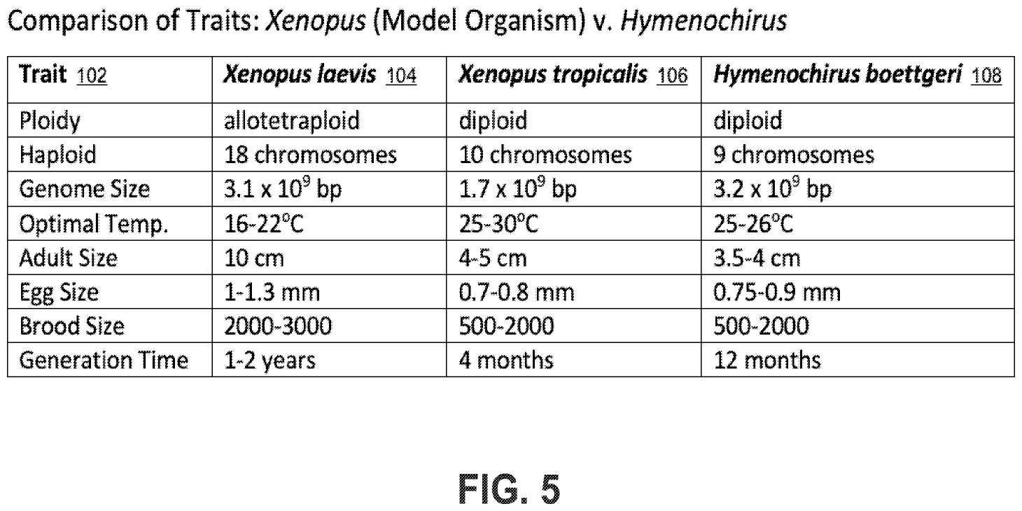

FIG. 5 depicts a chart comparing traits between a model organism and other amphibians, according to at least some embodiments disclosed herein. More specifically, FIG. 5 includes numerous columns, including: a first column 102 associated with a trait, a second column 104 associated with Xenopus laevis, a third column 106 associated with Xenopus tropicalis, and a fourth column 108 associated with Hymenochiru boettgeri.

As described herein, the pipid frog Xenopus is one of the favorite amphibian models of biologists, especially embryologists. The genus of Xenopus laevis (the African-clawed toad of the second column 104 of FIG. 5) is widely used in developmental biology. It can be induced to ovulate and mate any time of the year, following a simple injection of gonadotropic hormones. Xenopus (Silurana) tropicalis (of the third column 106 of FIG. 5) forms a separate, but evolutionarily related, lineage from X. laevis. Both species are highly similar in morphology, and share the same advantages with respect to embryological manipulation.

The African dwarf frog of the genus Hymenochirus is a type of aquatic frog native to parts of Equatorial Africa and is common in the pet trade and is often mistaken for the African clawed frog, a similar-looking frog in the same family. The African dwarf frog of the genus Hymenochirus is of particular interest, as the genus has never been modified, and Hymenochirus boettgeri is the only species of this genus where the genome has been sequenced, which occurred quite recently.

The first column 102 of FIG. 5 includes the following traits: ploidy (e.g., the number of complete sets of chromosomes in a cell, and hence the number of possible alleles for autosomal and pseudoautosomal genes), haploid, genome size (e.g., measured in bp, or base pairs), optimal temperature (measured in ° C.), adult size (measured in cm), egg size (measured in mm), brood size (which is variable across species and generally correlated with female body size), and generation time (measured in the number of months). As shown in FIG. 5, numerous traits associated with the third column 106 (associated with Xenopus tropicalis) are similar to traits associated with the fourth column 108 (associated with Hymenochiru boettgeri), such as ploidy, haploid, optimal temperature, adult size, egg size, and brood size, making Hymenochiru boettgeri an organism of interest for transgenic studies.

Preferably, the present invention provides a transgenic ornamental amphibian of the Hymenochirus genus that includes a golden/white variant, as such variant has emerged in the pet trade within the past few years and such variant allows for optimal visualization of fluorescence. There is no ownership restriction in the United States, as compared to Xenopus, which is a restricted species in some states in the United States. The fluorescent transgenic amphibian created by the instant invention is valuable in the market as a scientific research tool because the fluorescent transgenic amphibian can be used for embryonic studies, such as tracing cell lineage and cell migration. Cells from transgenic amphibians expressing fluorescent proteins can also be used as cellular and genetic markers in cell transplantation and nuclear transplantation experiments. Moreover, the fluorescent transgenic amphibians described herein may be used in the pet trade industry.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

### REFERENCES

The following references, to the extent that they provide exemplary procedural or other details supplementary to those set forth herein, are specifically incorporated herein by reference.
A. Chesneau, et al., “Transgenesis procedures in Xenopus,” Biology of the Cell, 2008, 100(9), Pages 503-521, doi:10.1042/BC20070148.
Kook-Ho Cho, et al., “Regulatory region of the vitellogenin receptor gene sufficient for high-level, germ line cell-specific ovarian expression in transgenic Aedes aegypti mosquitoes,” Insect Biochemistry and Molecular Biology, 2006, 36(4), Pages 273-281, doi: 10.1016/j.ibmb.2006.01.005.
N. D. L. Clark, et al., “The anatomy of conodonts,” Philosophical Transactions of the Royal Society B: Biological Sciences, 1993, 340(1294), Pages 405-421, doi: 10.1098/rstb.1993.0082.
Culp, et al., “Envelope glycoproteins from biologically diverse isolates of immunodeficiency viruses have widely different affinities for CD4,” Proceedings of the National Academy of Sciences of the United States of America, 1991, 88(2), Pages 512-516, doi: 10.1073/pnas.88.2.512.
Shao Jun Du, et al., “Growth enhancement in transgenic Atlantic salmon by the use of an “all fish” chimeric growth hormone gene construct,” Nature Biotechnology, 1992, 10, Pages 176-181, doi: 10.1038/nbt0292-176.
Maria Eckert, et al., “Agrobacterium tumefaciens-mediated transformation of Leptosphaeria spp. and Oculimacula spp. with the reef coral gene DsRed and the jellyfish gene gfp,” FEMS Microbiology Letters, 2005, 253, Pages 67-74, doi: 10.1016/j.femsle.2005.09.041.
J. W. Gordon, et al., “Genetic transformation of mouse embryos by microinjection of purified DNA,” Proceedings of the National Academy of Sciences of the United States of America, December 1980, 77(12), Pages 7380-7384, doi: 10.1073/pnas.77.12.7380.
J. W. Goron and F. H. Ruddle, “Integration and stable germ line transmission of genes injected into mouse pronuclei,” Science, December 1981, 214(4526), Pages 1244-1246, doi: 10.1126/science.6272397.
Hiroaki Inoue, et al., “High efficiency transformation of Escherichia coli with plasmids,” Gene, 1990, 96(1), Pages 23-28, doi: 10.1016/0378-1119(90)90336-P.
K. L. Kroll and E. Amaya, et al., “Transgenic Xenopus embryos from sperm nuclear transplantations reveal FGF signaling requirements during gastrulation,” Development, 1996, 122(3), Pages 173-3183, PMID: 8898230.
R. Lathe & J. J. Mullins, “Transgenic animals as models for human disease—report of an EC Study Group,” Transgenic Research, 1993, 2(5), Pages 286-299, doi: 10.1007/BF01968841.
M. V. Matz, et al., “Fluorescent proteins from nonbioluminescent Anthozoa species,” Nature Biotechnology, 1999, 17(10), Pages 969-73, doi: 10.1038/13657.
A. W. Murray, “Chapter 30: Cell cycle extracts,” Methods in Cell Biology, 1991 36, Pages 581-605, doi: 10.1016/50091-679X(08)60298-8.
Hajime Ogino, et al., “Highly efficient transgenesis in Xenopus tropicalis using I-SceI meganuclease,” Mechanisms of Development, 2006, 123(2), Pages 103-113, doi: 10.1016/j.mod.2005.11.006.
Richard D. Palmiter & Ralph L. Brinster, “Germ-line transformation of mice,” Annual Review of Genetics, 1986, 20, Pages 465-499, doi: 10.1146/annurev.ge.20.120186.002341.
R. D. Palmiter, et al., “Heterologous introns can enhance expression of transgenes in mice,” Proceedings of the National Academy of Sciences of the United States of America, 1991, 88(2), Pages 478-482, doi: 10.1073/pnas.88.2.478.
D. A. Powers, et al., “Electroporation: a method for transferring genes into the gametes of zebrafish (Brachydanio rerio), channel catfish (Ictalurus punctatus), and common carp (Cyprinus carpio),” Molecular Marine Biology and Biotechnology, 1992, 1(4-5), Pages 301-8, PMID: 1339228.
Sambrook et al., In: Molecular Cloning: A Laboratory Manual, 2^{nd} Ed., Cold Spring Harbor Laboratory Press, Cold Spring Harbor, N.Y., 1989.
Sambrook and Russell, Molecular Cloning: A Laboratory Manual, 3^{rd} Ed., Cold Spring Harbor Laboratory Press, 2001.
Abhimanyu Sarkar, et al., “Insulated piggyBac vectors for insect transgenesis,” BMC Biotechnology, 2006, 6, Page 27, doi: 10.1186/1472-6750-6-27.
F. Y. T. Sin, et al., “Electroporation of salmon sperm for gene transfer: efficiency, reliability, and fate of transgene,” Molecular reproduction and development, 2000, 56, Pages 285-288.
Sippel et al., In: The Regulatory Domain Organization of Eukaryotic Genomes: Implications For Stable Gene Transfer, Transgenic Animals, Grosveld and Kollias (Eds.), Academic Press, 1-26, 1992.
D. B. Sparrow, et al., “A simplified method of generating transgenic Xenopus,” Nucleic Acids Research, 2000, 28(4), Page E12, doi: 10.1093/nar/28.4.e12.
J. Szelei, et al., “Liposome-mediated gene transfer in fish embryos,” Transgenic Research, 1994, 3, Pages 116-119.
Jakub Tolar, et al., “Real-time in vivo imaging of stem cells following transgenesis by transposition,” Molecular Therapy, 2005, 12(1), Pages 42-48, doi: 10.1016/j.ymthe.2005.02.023.
Charline Walker and George Streisinger, “Induction of mutations by γ-rays in pregonial germ cells of zebrafish embryos,” Genetics, 1983, 103(1), Pages 125-136.
A. Wenck, et al., “Reef-coral proteins as visual, non-destructive reporters for plant transformation,” Plant Cell Reports, 2003, 22, Pages 244-251, doi: 10.1007/s00299-003-0690-x.
D. Werdien, et al., “FLP and Cre recombinase function in Xenopus embryos,” Nucleic Acids Research, 2001, 29(11), Pages E53-3, doi: 10.1093/nar/29.11.e53.
Mira Wouters, et al., “W^{ZsGreen}/+: a new green fluorescent protein knock-in mouse model for the study of KIT-expressing cells in gut and cerebellum,” Physiological Genomics, 2005, 22(3), Pages 412-421, doi: 10.1152/physiolgenomics.00105.2005.
Y. Xu, et al., “Fast skeletal muscle-specific expression of a zebrafish myosin light chain 2 gene and characterization of its promotor by direct injection into skeletal muscle,” DNA and Cell Biology, 1999, 18, Pages 85-95, doi: 10.1089/104454999315655.
Alexander V. Zelenin, et al., “The delivery of foreign genes into fertilized fish eggs using high-velocity microprojectiles,” FEBS Letters, 1991, 287(2), Pages 118-120, doi: 10.1016/0014-5793(91)80029-3.
Robert W. Zeller, et al., “Predictable mosaic transgene expression in ascidian embryos produced with a simple electroporation device,” Developmental Dynamics, 2006, 235(7), Pages 1921-1932, doi: 10.1002/dvdy.20815.
Hao Zhu and Leonard I. Zon, “Use of the DsRed fluorescent reporter in zebrafish,” Methods in Cell Biology, 2004, 76, Pages 3-12, doi: 10.1016/s0091-679x(04)76001-x.
Hao Zhu, et al., “Regulation of the lmo2 promoter during hematopoietic and vascular development in zebrafish,” Developmental Biology, 2005, 281(2), Pages 256-269, doi: 10.1016/j.ydbio.2005.01.034.

What is claimed is:

1. A transgenic fluorescent amphibian comprising in its genome:
(a) a transgene encoding a fluorescent protein, wherein the transgene is under the control of a ubiquitous promoter; and/or
(b) a transgene encoding a fluorescent protein, wherein the transgene is under the control of a tissue specific promoter; and/or
(c) one or more transgenes encoding one or more fluorescent proteins, wherein the transgenes are under the control of ubiquitous and/or tissue specific promoters; and
where said transgenic fluorescent amphibian exhibits a visible fluorescence of the body of said amphibian, optionally excluding eyes, and wherein said transgene(s) are chromosomally integrated.

2. The transgenic fluorescent amphibian of claim 1, wherein the fluorescent gene is selected from the group consisting of ZsGreen1, ZsYellow1, DsRed2, GFP, eGFP, YFP, eYFP, BFP, eBFP, CFP, eCFP, FP, AmCyan1, DsRed-Express, AsRed2, HcRed1,mPlum, mCherry, tdTomato, mStrawberry, J-Red, DsRed-monomer, morange, mKO, MCitrine, Venus, Ypet, EYFP, Emerald, CyPet, mCFPm, Cerulean, and T-Sapphire.

3. The transgenic fluorescent amphibian of claim 1, wherein the ubiquitous promoter is selected from the group consisting of promoters of acidic ribosomal phosphoprotein (ARP), α-catenin, β-catenin, γ-catenin, and EF-1 α.

4. The transgenic fluorescent amphibian of claim 1, wherein the tissue specific promoter is a muscle-specific promoter.

5. The transgenic fluorescent amphibian of claim 4, wherein the muscle-specific promoter is selected from the group consisting of: β-actin, desmin, dystrophin, myosin heavy chain, myosin light chain, MyoD, myogenin, muscle creatine kinase, β-sarcoglycan, serum response factor, and α-tropomyosin.

6. The transgenic fluorescent amphibian of claim 1, wherein the tissue specific promoter comprises a skin specific promoter.

7. The transgenic fluorescent amphibian of claim 1, wherein the tissue specific promoter comprises an eye specific promoter.

8. The transgenic fluorescent amphibian of claim 1, wherein the tissue specific promoter comprises a bone specific promoter.

9. The transgenic fluorescent amphibian of claim 1, wherein a species of an ornamental amphibian is selected from the group consisting of: Hymenochirus, sp., Hymenochirus boettgeri, Hymenochirus boulengeri, Hymenochirus curtipes, Hymenochirus feae, Xenopus, sp., Xenopus laevis, Xenopus tropicalis, Trachycephalus Resinfictrix, Anaxyrus americanus, Ambystoma mexicanum, Lepidobatrachus laevis, Melanophryniscus stelzneri, Typhlonectes compressicauda, Triturus cristatus, Bombina, sp., Cynops, sp., Salamandra, Cryptobranchidae, Hyla versicolor, Occidozyga lima, Cryptobranchus alleganiensis, Neurergus kaiseri, Siren intermedia, Necturus, sp., Ceratophryidae, sp., Lithobates grylio, Pyxicephalus, sp., Dendrobates, sp., Agalychnis callidryas, Typhlonectes natans, Ambystoma tigrinum, and Lictoria caerulea.

10. The transgenic fluorescent amphibian of claim 1, wherein a species of an ornamental amphibian is from the genus Hymenochirus.

11. The transgenic fluorescent amphibian of claim 10, wherein a species of an ornamental amphibian from the genus Hymenochirus is an albino variant.

12. The transgenic fluorescent amphibian of claim 10, wherein a species of an ornamental amphibian from the genus Hymenochirus is a leucistic variant.

13. A transgenic fluorescent amphibian of claim 1, further defined as a fertile, transgenic amphibian.

14. A transgenic fluorescent amphibian of claim 1, wherein the amphibian is homozygous for the integrated transgene(s).

15. A transgenic fluorescent amphibian of claim 1, wherein the amphibian is heterozygous for the integrated transgene(s).

16. A transgenic fluorescent amphibian of claim 1, wherein the integrated transgene(s) are heritable.

17. A method of producing a transgenic fluorescent ornamental amphibian that exhibits fluorescence, the method comprising:
(a) a cloning vector which comprises a transgene that contains a gene or gene that encodes for one or more fluorescent proteins, wherein the gene or genes is under the control of a ubiquitous promoter and/or a tissue specific promoter, and wherein the terminator sequence is comprised of one or more polyadenylation signals; and
(b) the transgene is chromosomally integrated into the genome of an amphibian to create a transgenic fluorescent amphibian that exhibits a visible fluorescence of the body, optionally excluding eyes.

18. The method of claim 17, wherein the fluorescent gene or genes is selected from the group consisting of ZsGreen1, ZsYellow1, DsRed2, GFP, eGFP, YFP, eYFP, BFP, eBFP, CFP, eCFP, FP, AmCyan1, DsRed-Express, AsRed2, HcRed1,mPlum, mCherry, tdTomato, mStrawberry, J-Red, DsRed-monomer, morange, mKO, MCitrine, Venus, Ypet, EYFP, Emerald, CyPet, mCFPm, Cerulean, and T-Sapphire.

19. The method of claim 17, wherein the ubiquitous promoter is selected from the group consisting of: promoters of acidic ribosomal phosphoprotein (ARP), α-catenin, β-catenin, γ-catenin, and EF-1 α.

20. The method of claim 17, wherein the tissue specific promoter is a muscle-specific promoter.

21. The method of claim 20, wherein the muscle-specific promoter is selected from the group consisting of: β-actin, desmin, dystrophin, myosin heavy chain, myosin light chain, MyoD, myogenin, muscle creatine kinase, β-sarcoglycan, serum response factor, and α-tropomyosin.

22. The method of claim 17, wherein the cloning vector and transgene comprises at least one polyadenylation signal or two polyadenylation signals positioned in tandem.

23. The method of claim 22, wherein the polyadenylation signal(s) are viral polyadenylation signals.

24. The method of claim 23, wherein the polyadenylation signal(s) are SV40 polyadenylation sequences.

25. The method of claim 17, wherein a species of an ornamental amphibian is selected from the group consisting of: Hymenochirus, sp., Hymenochirus boettgeri, Hymenochirus boulengeri, Hymenochirus curtipes, Hymenochirus feae, Xenopus laevis, Trachycephalus Resinfictrix, Anaxyrus americanus, Ambystoma mexicanum, Lepidobatrachus laevis, Melanophryniscus stelzneri, Typhlonectes compressicauda, Xenopus, sp., Triturus cristatus, Bombina, sp., Cynops, sp., Salamandra, Cryptobranchidae, Hyla versicolor, Occidozyga lima, Cryptobranchus alleganiensis, Neurergus kaiseri, Siren intermedia, Necturus, sp., Ceratophryidae, sp., Lithobates grylio, Pyxicephalus, sp., Dendrobates, sp., Agalychnis callidryas, Typhlonectes natans, Ambystoma tigrinum, Xenopus tropicalis, and Lictoria caerulea.

26. The method of claim 17, wherein a species of an ornamental amphibian is from the genus Hymenochirus.

27. The method of claim 26, wherein a species of an ornamental amphibian from the genus Hymenochirus is an albino variant.

28. The method of claim 26, wherein a species of an ornamental amphibian from the genus Hymenochirus is a leucistic variant.

29. A method of producing a population of transgenic fluorescent amphibians that exhibit fluorescence, the method comprising:
(a) obtaining an amphibian that exhibits fluorescence and comprises one or more chromosomally integrated transgenes that encode for one or more fluorescent proteins; and
(b) breeding the obtained amphibian with a second amphibian to create progeny containing the heritable chromosomally integrated transgene(s).

30. The method of claim 29, wherein the second amphibian is a transgenic amphibian.

31. The method of claim 29, wherein the second amphibian is a non-transgenic amphibian.

32. The method of claim 29, wherein the progeny of the transgenic fluorescent amphibian exhibits a visible fluorescence of the body, optionally excluding eyes.

33. A method of providing a transgenic amphibian to the ornamental amphibian market, comprising the steps of:
(a) breeding a transgenic amphibian comprising a chromosomally integrated transgene encoding one or more fluorescent protein; or
(b) breeding a progeny amphibian, wherein the amphibian comprises a chromosomally integrated transgene encoding one or more fluorescent protein; and
(c) distributing the amphibian to the ornamental amphibian market, wherein the amphibian is distributed by a breeder.

34. The method of claim 33, wherein a species or a progeny of an ornamental amphibian is selected from the group consisting of: Hymenochirus, sp., Hymenochirus boettgeri, Hymenochirus boulengeri, Hymenochirus curtipes, Hymenochirus feae, Xenopus laevis, Trachycephalus Resinfictrix, Anaxyrus americanus, Ambystoma mexicanum, Lepidobatrachus laevis, Melanophryniscus stelzneri, Typhlonectes compressicauda, Xenopus, sp., Triturus cristatus, Bombina, sp., Cynops, sp., Salamandra, Cryptobranchidae, Hyla versicolor, Occidozyga lima, Cryptobranchus alleganiensis, Neurergus kaiseri, Siren intermedia, Necturus, sp., Ceratophryidae, sp., Lithobates grylio, Pyxicephalus, sp., Dendrobates, sp., Agalychnis callidryas, Typhlonectes natans, Ambystoma tigrinum, Xenopus tropicalis, and Lictoria caerulea.

35. The method of claim 33, wherein a species or a progeny of an ornamental amphibian is derived from the genus Hymenochirus.

36. The method of claim 33, wherein a species or a progeny of an ornamental amphibian derived from the genus Hymenochirus is an albino variant.

37. The method of claim 33, wherein a species or a progeny of an ornamental amphibian derived from the genus Hymenochirus is a leucistic variant.

38. The method of claim 33, wherein the amphibians being distributed by a breeder are further distributed to a commercial distributor.

39. The method of claim 33, wherein the amphibians being distributed by a breeder or a commercial distributor are further distributed to a retailer.

40. The method of claim 39, wherein the retailer is a multi-product retailer having an ornamental aquatics department.
